(19) United States
(12) Patent Application Publication
(10) Pub. No.: US 2015/0037424 A1
(43) Pub. Date: Feb. 5, 2015

(54) SUSTAINED RELEASE ORAL SOLID PREPARATION

(71) Applicant: OTSUKA PHARMACEUTICAL CO., LTD., Chiyoda-ku, Tokyo (JP)

(72) Inventors: Robert A. Forbes, Princeton, NJ (US); Suresh Mallikaarjun, Rockville, MD (US); Arash Raoufinia, Rockville, MD (US); Ron Casey, Rockville, MD (US); Junichi Jinno, Osaka-shi (JP); Hiroyuki Nagao, Osaka-shi (JP); Donald Diehl, New Fairfield, CT (US); Erik Pecorelli, Loveland, OH (US); Anthony Carpanzano, New Milford, CT (US)

(73) Assignee: OTSUKA PHARMACEUTICAL CO., LTD., Chiyoda-ku, Tokyo (JP)

(21) Appl. No.: 14/383,401

(22) PCT Filed: Mar. 6, 2013
(86) PCT No.: PCT/JP2013/056881

§ 371 Date: Sep. 5, 2014

Related U.S. Application Data
(60) Provisional application No. 61/607,291, filed on Mar. 6, 2012.

(51) Int. Cl. A61K 47/36; A61K 9/20; A61K 47/02; A61K 31/496

(52) U.S. Cl. CPC A61K 47/36; A61K 31/496; A61K 9/20; A61K 47/02
USPC 424/497; 514/253.07; 424/490

(57) ABSTRACT

Provided is a sustained release oral solid preparation comprising aripiprazole or a salt thereof as an active ingredient described below, and a method for producing the sustained release oral solid preparation.

A sustained release oral solid preparation comprising aripiprazole or a salt thereof and a sustained release excipient, the sustained release excipient comprising a gelling agent; at least one inert pharmaceutical diluent selected from the group consisting of monosaccharides, disaccharides, polyhydric alcohols, and mixtures thereof; and a pharmaceutically acceptable cationic cross-linking agent capable of crosslinking with the gelling agent and increasing the gel strength when the sustained release oral solid preparation is exposed to an environmental fluid, the gelling agent comprising xanthan gum and locust bean gum, the ratio of the xanthan gum to the locust bean gum in the gelling agent being about 1:1 to 1:3 by weight, the ratio of the inert pharmaceutical diluent to the gelling agent being about 1:1 to 1:2 by weight.

### TECHNICAL FIELD

The present invention relates to a sustained release oral solid preparation comprising aripiprazole or a salt thereof.

### BACKGROUND ART

Aripiprazole is a compound represented by the following formula (I):

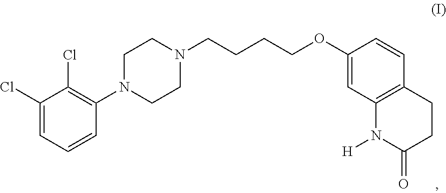 [SMILES: O=C1CCc2ccc(OCCCCN3CCN(c4cccc(Cl)c4Cl)CC3)cc2N1]

and is known as an atypical antipsychotic drug effective for the treatment of schizophrenia (U.S. Pat. No. 5,006,528).

Aripiprazole is used in Japan as an active ingredient of a medicinal product effective for ameliorating schizophrenia symptoms and manic symptoms in bipolar disorder. Such a medicinal product has been available from Otsuka Pharmaceutical, Co., Ltd.

Aripiprazole and salts thereof are known as active ingredients for treating disorders of the central nervous system (CNS) associated with 5-HT_{1A} receptor subtype, and are also known to be effective for, for example, the diseases disclosed in Japanese Patent No. 4178032.

For patients suffering from an aforementioned disorder of the central nervous system (CNS disease), there is a need for sustained release oral preparations, in order to, for example, improve medication compliance and quality of life (QOL) in pediatric patients.

Hitherto, various sustained release preparations have been known. However, certain moderately to poorly soluble drugs present formulation difficulties that render them inapplicable for sustained release preparations that might be suitable for, for example, relatively soluble drugs. It is often not possible to readily predict whether a particular sustained release oral solid preparation will provide the desired sustained release for a relatively insoluble drug, and it has generally been found to be necessary to carry out considerable experimentation to obtain sustained release oral solid preparations that have desired pharmacokinetics when ingested.

Aripiprazole is very poorly soluble, and often exhibits poor pharmacokinetics when incorporated into a sustained release oral solid preparation. Therefore, it has been an urgent necessity for doctors engaged in medical practice to develop a safe and persistent oral preparation using aripiprazole.

### SUMMARY OF INVENTION

### Technical Problem

An object of the present invention is to provide a sustained release oral solid preparation comprising aripiprazole or a salt thereof as an active ingredient, and a method for producing the sustained release oral solid preparation.

### Solution to Problem

The sustained release oral solid preparation of the present invention comprises, at a specific weight ratio, aripiprazole or a salt thereof; a gelling agent; at least one inert pharmaceutical diluent selected from the group consisting of monosaccharides, disaccharides, polyhydric alcohols, and mixtures thereof; and a pharmaceutically acceptable cationic cross-linking agent capable of crosslinking with the gelling agent and increasing the gel strength when the oral solid preparation is exposed to an environmental fluid. The present inventors found that such a sustained release oral solid preparation of the present invention can provide sustained release of aripiprazole or a salt thereof when exposed to an environmental fluid, and is useful as, in particular, a once-weekly (QW) oral preparation.

The present invention has been accomplished based on these findings and further research, and includes, for example, the subject matter shown below.

Item 1. A sustained release oral solid preparation comprising aripiprazole or a salt thereof, and a sustained release excipient,

the sustained release excipient comprising
a gelling agent comprising xanthan gum and locust bean gum;
at least one inert pharmaceutical diluent selected from the group consisting of monosaccharides, disaccharides, polyhydric alcohols, and mixtures thereof; and,
a pharmaceutically acceptable cationic cross-linking agent capable of crosslinking with the gelling agent and increasing the gel strength when the sustained release oral solid preparation is exposed to an environmental fluid,
the ratio of the xanthan gum to the locust bean gum in the gelling agent being about 1:1 to 1:3 by weight,
the ratio of the inert pharmaceutical diluent to the gelling agent being about 1:1 to 1:2 by weight.

Item 2. The sustained release oral solid preparation according to Item 1, further comprising hypromellose, wherein the ratio of the hypromellose to the aripiprazole or a salt thereof is about 1:0.1 to 1:5, preferably about 1:0.4 to 1:2.8 by weight.

Item 3. The sustained release oral solid preparation according to Item 1 or 2, wherein the cationic cross-linking agent is at least one salt selected from the group consisting of sulfate, chloride, borate, carbonate, phosphate, bromide, citrate, acetate, and lactate, and wherein the salt is an alkali metal salt or an alkaline earth metal salt.

Item 4. The sustained release oral solid preparation according to any one of Items 1 to 3, wherein the cationic cross-linking agent comprises calcium sulfate.

Item 5. A sustained release oral solid preparation comprising aripiprazole or a salt thereof, and a sustained release excipient,

the sustained release excipient comprising
a gum comprising a combination of xanthan gum with locust bean gum;
a sugar alcohol (preferably mannitol, xylitol, or erythritol, and more preferably mannitol); and
an inorganic or organic salt of an alkali metal and/or an alkaline earth metal (preferably at least one member selected from calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, and sodium fluoride, more preferably calcium sulfate or sodium chloride, and still more preferably calcium sulfate),
the ratio of the xanthan gum to the locust bean gum being about 1:1 to 1:3 by weight, and
the ratio of the sugar alcohol to the gum being within the range of about 1:1 to 1:2 by weight.

Item 6. The sustained release oral solid preparation according to Item 5, further comprising hypromellose.

Item 7. The sustained release oral solid preparation according to Item 6, wherein the sustained release oral solid preparation comprises about 45 to 85 weight % of aripiprazole or a salt thereof, and hypromellose in an amount satisfying the following inequality:

−0.733x+71≦y≦−0.733x+77,

wherein x represents a proportion (wt %) of the aripiprazole or a salt thereof, and y represents a proportion (wt %) of the hypromellose.

Item 8. The sustained release oral solid preparation according to any one of Items 1 to 7, which is a tablet.

Item 9. The sustained release oral solid preparation according to any one of Items 1 to 8, wherein at least part of a surface of the sustained release oral solid preparation is coated with an enteric material, and wherein the sustained release oral solid preparation coated with the enteric material has a weight increased by about 1 to 20 wt %, preferably about 6 wt %, relative to the sustained release oral solid preparation before coating.

Item 10. The sustained release oral solid preparation according to Item 9, wherein the enteric material comprises a methacrylic acid copolymer.

Item 11. The sustained release oral solid preparation according to Item 1, 2, 3, 4, 8, 9, or 10, wherein the content of the cationic cross-linking agent is about 0.5 to 20 wt % of the sustained release excipient.

Item 12. A method for producing a sustained release oral solid preparation, comprising

mixing aripiprazole or a salt thereof with a sustained release excipient,

the sustained release excipient comprising
about 3.0 to 98.5 wt % of a gum comprising xanthan gum and locust bean gum;
about 1.0 to 89 wt % of a sugar alcohol (preferably mannitol, xylitol or erythritol, and more preferably mannitol); and
about 0.5 to 20 wt % of an inorganic or organic salt of an alkali metal and/or an alkaline earth metal (preferably at least one member selected from calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, and sodium fluoride, more preferably calcium sulfate or sodium chloride, and still more preferably calcium sulfate),

the ratio of the xanthan gum to the locust bean gum being about 1:1 to 1:3 by weight, and

the ratio of the sugar alcohol to the gum being within the range of about 1:1 to 1:2 by weight.

The sustained release oral solid preparation obtained by the production method of Item 12 preferably maintains a therapeutically effective blood level of the aripiprazole or a salt thereof for at least 12 hours, more preferably at least about one week, up to about 2 weeks when exposed to an environmental fluid.

Item 13. A method for producing a sustained release oral solid preparation, comprising:

preparing a sustained release excipient comprising
about 3.0 to 98.5 wt % of a gelling agent comprising xanthan gum and locust bean gum;
about 1.0 to 89 wt % of an inert pharmaceutical diluent; and
about 0.5 to 20 wt % of a cationic cross-linking agent capable of crosslinking with the gelling agent and increasing the gel strength when the sustained release oral solid preparation is exposed to an environmental fluid,
the ratio of the xanthan gum to the locust bean gum being about 1:1 to 1:3 by weight; and

mixing the sustained release excipient with aripiprazole or a salt thereof to prepare a sustained release oral solid preparation,

wherein when the sustained release oral solid preparation is exposed to an environmental fluid, the preparation maintains a therapeutically effective blood level of the aripiprazole or a salt thereof for at least 12 hours, preferably at least about one week, up to about 2 weeks.

Item 14. The method for producing a sustained release oral solid preparation according to Item 12 or 13, wherein the sustained release excipient is mixed with aripiprazole or a salt thereof so that the produced sustained release oral solid preparation comprises about 45 to 85 wt % of aripiprazole or a salt thereof, and hypromellose in an amount satisfying the following inequality:

−0.733x+71≦y≦−0.733x+77,

wherein x represents the proportion (wt %) of the aripiprazole or a salt thereof, and y represents the proportion (wt %) of the hypromellose.

Item 15. A method for treating a central nervous system disease, comprising orally administering the sustained release oral solid preparation of any one of Items 1 to 11 to a patient suffering from a central nervous system disease.

This application claims the priority of U.S. Provisional Application No. 61/607,291 filed Mar. 6, 2012, the contents of the specification and/or the drawings of the provisional application are incorporated herein by reference.

The expression “to comprise” used herein also includes the meanings of “to essentially consist of” and “to consist of.”

### Advantageous Effects of Invention

According to the sustained release oral solid preparation of the present invention, even when the preparation is administered at a high dose, an initial “burst” of the drug release from the sustained release oral solid preparation at the time the sustained release oral solid preparation is exposed to an aqueous solution or gastrointestinal fluid can be prevented, and an unnecessary increase in the blood level peak of the aripiprazole or a salt thereof, i.e., the active ingredient, can be suppressed.

Further, the effective blood level of the aripiprazole or a salt thereof can be maintained for 12 hours, more preferably 24 hours, and most preferably 1 week, up to about 2 weeks.

Similar to conventional aripiprazole tablets, the sustained release preparation of the present invention is effective for ameliorating schizophrenia symptoms and manic symptoms in bipolar disorder, as well as the diseases disclosed in Japanese Patent No. 4178032, described above. The sustained release oral solid preparation of the present invention is particularly effective for Tourette's syndrome, which is a CNS disease. It is particularly preferable that the preparation of the invention contains 150 to 300 mg of aripiprazole when used for schizophrenia symptoms.

Aripiprazole having any crystal form may be used for the preparation of the invention; however, it is most preferable that aripiprazole anhydride crystals B disclosed in Japanese Patent No. 3760264 be used. The disclosures of this patent document are incorporated herein by reference.

### BRIEF DESCRIPTION OF DRAWINGS

FIG. 1 is a graph showing average blood level versus time profiles of aripiprazole in test subjects after administration of the oral preparation of Example 2 or 3 in the fasting state in Test Example 1. In the figure, the black squares (77.5 mg) show the results of Example 2 using Tablet 2, and the black triangles (110 mg) show the results of Example 3 using Tablet 3.

FIG. 2 is a graph showing average blood level versus time profiles of aripiprazole in test subjects after administration of the oral preparation of Example 1 after a meal in Test Example 2.

### DESCRIPTION OF EMBODIMENTS

The sustained release oral solid preparation of the present invention comprises aripiprazole or a salt thereof, and a sustained release excipient.

The sustained release excipient comprises a gelling agent; at least one inert pharmaceutical diluent selected from the group consisting of monosaccharides, disaccharides, polyhydric alcohols, and mixtures thereof; and a pharmaceutically acceptable cationic cross-linking agent capable of crosslinking with the gelling agent and increasing the gel strength when the sustained release oral solid preparation is exposed to an environmental fluid.

The term “sustained release” used in this specification means that the drug is released from the sustained release oral solid preparation at a controlled rate such that therapeutically beneficial blood levels (but below toxic levels) of the active ingredient is maintained over an extended period of time, e.g., providing a dosage form that can maintain the effective blood level of the drug for 12 hours or 24 hours, most preferably 1 week.

The term “pharmacokinetics” (PK) refers to the disposition of drugs in the body. The study of how a drug is processed (absorbed, distributed, metabolized, and excreted) by the body, as well as the study of enzymes, transporters, etc., involved therein, reveals the disposition of drugs in the body. The term “environmental fluid” refers to a fluid that presents in the environment of the sustained release oral solid preparation when the sustained release oral solid preparation is taken by a subject. For example, the term “environmental fluid” refers to water ingested with the preparation or to a fluid secreted by digestive organs. The term may also be referred to as “dissolution fluid.” Specific examples of environmental fluids include water, an aqueous solution, saliva, gastrointestinal fluid, and the like.

Hereinafter, each component is described in detail.

The gelling agent used in the present invention comprises xanthan gum and locust bean gum.

Xanthan gum is a high molecular weight (>10⁶) polysaccharide with a structure having a main chain of glucose to which side chains of mannose, glucuronic acid, and pyruvic acid are attached.

Locust bean gum is a polysaccharide with a structure having a main chain of mannose and a side chain of galactose. Locust bean gum is a kind of galactomannans. Galactomannans are polysaccharides composed mainly of mannose and galactose.

A combination of xanthan gum with a galactomannan can be used as a gelling agent; however, the present invention particularly uses locust bean gum among galactomannans. Galactomannans, which have higher proportions of unsubstituted mannose regions, have been found to achieve more interaction with xanthan gum. Locust bean gum, which has a higher ratio of mannose to galactose, as compared to other galactomannans such as guar gum and hydroxypropyl guar gum, is particularly preferred.

The gelling agent used in the present invention may contain one or more other polysaccharides (e.g., homopolysaccharide gum), in addition to xanthan gum and locust bean gum. The gelling agent used in the present invention is particularly preferably a gum comprising a combination of xanthan gum with a galactomannan.

The ratio of the xanthan gum to the locust bean gum is about 1:1 to 1:3, and is more preferably about 1:1 to 1:2, by weight. The gelling agent is contained in the sustained release excipient in a proportion of preferably about 3.0 to 98.5 wt %, more preferably about 10 to 98.5 wt %, still more preferably about 30 to 70 wt %, and even more preferably about 40 to 60 wt %.

The gelling agent is contained in the sustained release oral solid preparation in a proportion of preferably about 0.5 to 50 wt %, more preferably about 1 to 50 wt %, and still more preferably about 2 to 30 wt %. In particular, the proportion of the gelling agent in the sustained release oral solid preparation is preferably about 1 to 20 wt % is preferable, more preferably about 1 to 10 wt %, and still more preferably about 1 to 5 wt %.

The ratio of the gelling agent to the aripiprazole or a salt thereof (gelling agent:aripiprazple or a salt thereof) is preferably about 1:1 to 1:100, more preferably about 1:1 to 1:50, still more preferably about 1:2 to 1:30, and even more preferably about 1:5 to 1:25, by weight.

The inert pharmaceutical diluent preferably comprises at least one pharmaceutically acceptable saccharide selected from the group consisting of monosaccharides, disaccharides, polyhydric alcohols, and mixtures of any of the foregoing. Specific examples of inert pharmaceutical diluents include sucrose, dextrose, lactose, microcrystalline cellulose, fructose, xylitol, sorbitol, mannitol, erythritol, maltitol, reduced palatinose, mixtures thereof, and the like. These inert pharmaceutical diluents may be used singly or in a combination of two or more. As an inert pharmaceutical diluent, a sugar alcohol is preferable among saccharides, with mannitol, xylitol, and erythritol being more preferable, and with mannitol being particularly preferable.

The ratio of the inert pharmaceutical diluent to the gelling agent is about 1:1 to 1:2 by weight.

The inert pharmaceutical diluent is contained in the sustained release excipient in a proportion of preferably about 1.0 to 89 wt %, more preferably about 5 to 50 wt %, and still more preferably about 30 to 50 wt %.

The inert pharmaceutical diluent is contained in the sustained release oral solid preparation in a proportion of preferably about 0.5 to 80 wt %, more preferably about 1 to 80 wt %, still more preferably about 2.0 to 10 wt %, and even more preferably about 2.0 to 5 wt %.

The ratio of the inert pharmaceutical diluent to the aripiprazole or a salt thereof (inert pharmaceutical diluent:aripiprazole or a salt thereof) is preferably about 1:3 to 1:125, more preferably about 1:3 to 1:60, still more preferably about 1:3 to 1:50, even more preferably about 1:4 to 1:30, and particularly preferably about 1:10 to 1:30, by weight.

The sustained release excipient consisting only of the gelling agent is not sufficient to provide desired sustained release of an insoluble drug (aripiprazole or a salt thereof), nor is it sufficient to prevent an initial “burst” of drug release from the sustained release oral solid preparation when the sustained release oral solid preparation is exposed to a fluid in an environment of use (environmental fluid), e.g., an aqueous solution or gastrointestinal fluid.

This problem has been overcome by the present invention, and according to one aspect of the present invention, by incorporating a cationic cross-linking agent into the sustained release excipient, the gel strength of the sustained release oral solid preparation can be significantly increased.

The cationic cross-linking agent used in the present invention is a pharmaceutically acceptable cationic cross-linking agent capable of increasing the gel strength when the sustained release oral solid preparation is exposed to an environmental fluid. Specific examples of the cationic cross-linking agents include salts that generate monovalent or multivalent metal cations. Examples of preferable salts include inorganic and organic salts of various alkali metals and/or alkaline earth metals. Examples of the inorganic salts include alkali metal and/or alkaline earth metal sulphates, chlorides, borates, carbonates, phosphates, and bromides. Examples of the organic salts include alkali metal and/or alkaline earth metal citrates, acetates, lactates, and the like. Sodium, potassium, etc., are preferable as the alkali metal. Magnesium, calcium, etc., are preferable as the alkaline earth metal. Specific examples of suitable cationic cross-linking agents include calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, and sodium fluoride. Of these, preferable cross-linking agents are salts that generate divalent or monovalent cations. A preferable salt among these is calcium sulfate or sodium chloride, with calcium sulfate being particularly preferable. The cationic cross-linking agents may be used singly or in a combination of two or more. The cationic cross-linking agents of the present invention are incorporated in an amount effective to obtain a desirable gel strength. In preferred embodiments, the content of the cationic cross-linking agent is preferably about 0.1 to 20 wt %, more preferably about 0.5 to 20 wt %, still more preferably about 0.5 to 5.0 wt %, and even more preferably 0.5 to 2.0 wt %, of the sustained release oral solid preparation.

The ratio of the cationic cross-linking agent to the gelling agent (cationic cross-linking agent:gelling agent) is adjusted according to the components of the cationic cross-linking agent and gelling agent to be specifically used. For example, the ratio thereof is preferably about 1:1 to 1:10, and more preferably about 1:2 to 1:7, by weight.

The cationic cross-linking agent is contained in the sustained release excipient in a proportion of preferably about 0.5 to 20 wt %, more preferably about 1 to 20 wt %, and still more preferably about 5 to 15 wt %.

The ratio of the cationic cross-linking agent to the aripiprazole or a salt thereof is preferably about 1:10 to 1:500, more preferably about 1:10 to 1:250, still more preferably about 1:10 to 1:150, even more preferably about 1:15 to 1:110, and most preferably about 1:30 to 1:110, by weight.

Although it is not particularly limited, one of the most preferable examples of the sustained release excipient used in the present invention is an excipient comprising mannitol, calcium sulfate, and a gum comprising a combination of xanthan gum with locust bean gum. Here, the ratio of the xanthan gum to the locust bean gum is about 1:1 to 1:3 by weight, and the ratio of the mannitol and the gum is within the range of about 1:1 to 1:2 by weight.

The sustained release oral solid preparation of the present invention further comprising hypromellose (another name of hydroxypropyl methylcellulose (HPMC)) can maintain the effective blood level for about 1 week, up to about 2 weeks.

Specifically, in a preparation containing aripiprazole or a salt thereof and the above-described specific sustained release excipient, the sustained release property of aripiprazole is improved; further incorporation of hypromellose into the preparation further improves the sustained release property of aripiprazole.

The ratio of hypromellose to aripiprazole or a salt thereof is preferably about 1:0.1 to 1:10, more preferably about 1:0.1 to 1:5, and even more preferably about 1:0.4 to 1:2.8, by weight.

Hypromellose is contained in the sustained release oral solid preparation in a proportion of preferably about 10 to 60 wt %, more preferably about 20 to 60 wt %, and still more preferably about 20 to 40 wt %.

The form of the sustained release oral solid preparation of the present invention is not limited. Examples thereof include a powder, a granule, a tablet, and the like, with a tablet being preferable. The tablet may be a coated tablet. Specifically, the tablet includes not only uncoated tablets but also coated tablets. For example, chewable tablets, oral-disintegrating tablets, etc., are also included therein.

The sustained release oral solid preparation of the present invention may further comprise a pharmaceutical lubricant. In particular, the sustained release excipient may further comprise a pharmaceutical lubricant. The pharmaceutical lubricant may be added to the components of the sustained release excipient at the time the drug is added, or in any event prior to compression into the sustained release oral solid preparation. Examples of pharmaceutical lubricants include generally accepted pharmaceutical lubricants such as calcium soap or magnesium soap. Examples of lubricants include magnesium stearate, sodium stearyl fumarate, and the like. Sodium stearyl fumarate is a particularly preferable lubricant.

The lubricant is contained in the sustained release oral solid preparation in a proportion of preferably about 1 to 20 wt %, more preferably about 1 to 10 wt %, and still more preferably about 1.5 to 5.0 wt %.

The sustained release oral solid preparation of the present invention may further comprise a pharmaceutical superplasticizer. A superplasticizer particularly reduces the contacts between crystals with high hygroscopic property to prevent aggregation and crosslinking from occurring. A superplasticizer is therefore added for the purpose of increasing the flowability (in particular, for the purpose of increasing the flowability of a powder for tablet). Preferable examples of superplasticizers include fine silica (silicon dioxide).

The superplasticizer is contained in the sustained release oral solid preparation in a proportion of, for example, preferably about 0.1 to 2 wt %, more preferably about 0.2 to 1 wt %, and still more preferably about 0.3 to 0.7 wt %.

The sustained release oral solid preparation of the present invention may further contain various other pharmaceutically acceptable additives, as long as the effects of the present invention are not impaired.

Further, a coating may be formed on the sustained release oral solid preparation of the present invention. According to the present invention, in particular, the sustained release oral solid preparation is preferably coated with an enteric material (an enteric-coated sustained release oral solid preparation), because the effective blood level of the active ingredient, i.e., the aripiprazole or a salt thereof, can thereby be maintained for about 1 week, up to about 2 weeks. Further, a color coating may also be formed on the preparation of the invention.

When the sustained release oral solid preparation is a coated preparation, the proportions of the components contained in the preparation refer to the proportions relative to the sustained release oral solid preparation before the coating is formed thereon (uncoated preparation). For example, when the sustained release oral solid preparation is coated with an enteric material described below, the proportions relative to the sustained release oral solid preparation refer to the proportions relative to the sustained release oral solid preparation before an enteric material is coated thereon (uncoated preparation).

Examples of enteric materials (an enteric coating agent) optionally used in the present invention include methacrylic acid copolymer, cellulose acetate phthalate, hydroxypropyl methylcellulose phthalate, polyvinyl acetate phthalate, shellac, hydroxypropyl methylcellulose succinate, cellulose trimellitate acetate, and mixtures of any of the foregoing. Particularly preferred is a methacrylic acid copolymer. A copolymer obtained by copolymerization using a monomer mixture comprising ethyl acrylate and methacrylic acid is preferable as a methacrylic acid copolymer. Of these, a copolymer of ethyl acrylate and methacrylic acid is preferable. Among copolymers of ethyl acrylate and methacrylic acid, a copolymer obtained by copolymerization at a molar ratio of ethyl acrylate to methacrylic acid of about 2:1 to 1:2 is preferable, and a copolymer obtained by copolymerization at a molar ratio thereof of about 1:1 is particularly preferable. A commercially available product can also be used as a methacrylic acid copolymer. Specific examples thereof include Eudragit (Evonik Degussa), POLYQUID PA-30 (Sanyo Chemical Industries, Ltd.), and the like.

Enteric materials (enteric coating agents) may be used singly or in a combination of two or more.

The content of the enteric material is preferably about 3 to 10 wt %, and particularly preferably about 5 to 7 wt %, of the entire sustained release oral solid preparation coated with an aforementioned enteric material.

The sustained release oral solid preparations coated with an aforementioned enteric material preferably has a weight increased by preferably about 1 to 20 wt %, more preferably about 1 to 10 wt %, still more preferably about 6 to 8 wt %, relative to the sustained release oral solid preparations before coating.

The amount of the aripiprazole or a salt thereof contained in the sustained release oral solid preparation is preferably within the range of about 1 to 350 mg, more preferably about 1 to 250 mg, still more preferably about 1 to 200 mg, even more preferably about 20 to 150 mg. Particularly preferably, the amount of the aripiprazole or a salt thereof contained in the sustained release oral solid preparation is about 50 to 350 mg.

The properties of the sustained release excipient contained in the sustained release oral solid preparation of the present invention are dependent in part on the individual properties of the xanthan gum and locust bean gum, in terms of polymer solubility, glass transition temperatures etc.; and are also dependent on the synergism between both xanthan gum and locust bean gum, and between the xanthan gum, locust bean gum and an inert pharmaceutical diluent (e.g., inert saccharide constituents, preferably sugar alcohol) in modifying interactions between the dissolution fluid and excipient.

According to the sustained release oral solid preparation of the present invention, even when the preparation is administered at a high dose, an initial “burst” of the drug release from the sustained release oral solid preparation at the time the sustained release oral solid preparation is exposed to a dissolution fluid (e.g., an aqueous solution or gastrointestinal fluid) can be prevented, and an unnecessary increase in the blood level peak of the aripiprazole or a salt thereof, i.e., the active ingredient, can be suppressed. Further, the effective blood level of the aripiprazole or a salt thereof can be maintained for 12 hours, more preferably 24 hours, and even more preferably more than 1 week, up to about 2 weeks.

One of the most preferable forms of the sustained release oral solid preparation of the present invention is, but is not particularly limited to, a tablet in which an uncoated tablet containing aripiprazole or a salt thereof, a sustained release excipient, hypromellose, a superplasticizer (in particular fine silica), and a lubricant (in particular sodium stearyl fumarate) is coated with an enteric coating agent containing a methacrylic acid copolymer. Here, the sustained release excipient comprises mannitol, calcium sulfate, and a gum comprising a combination of xanthan gum with locust bean gum. The ratio of the xanthan gum to the locust bean gum is about 1:1 to 1:3 by weight, and the ratio of the mannitol to the gum is within the range of about 1:1 to 1:2 by weight. (Hereinafter this oral solid preparation is also referred to as “optimum dosage form preparation example 1”).

When the sustained release oral solid preparation of the present invention comprises hypromellose, the proportion of hypromellose is preferably adjusted according to the proportion of aripiprazole or a salt thereof. The effects of the present invention (the sustained release property of aripiprazole or a salt thereof) can thereby be better exerted. In particular, the adjustment of the proportion of hypromellose is preferably performed to obtain a preparation that satisfies the target PK profile of aripiprazole for Tourette's syndrome (in particular, pediatric Tourette's syndrome). In the optimum dosage form preparation example 1, the effects of the present invention can be better exerted by adjusting the proportion of hypromellose as above. Regarding the adjustment of the proportion of hypromellose, specifically, when the sustained release oral solid preparation comprises aripiprazole or a salt thereof in a proportion of about 45 to 85 wt % (preferably about 50 to 80 wt %), hypromellose is preferably contained therein in a proportion satisfying the following inequality:

−0.733x+71≦y≦−0.733x+77,

wherein x represents the proportion (wt %) of the aripiprazole or a salt thereof in the sustained release oral solid preparation, and y represents the proportion (wt %) of the hypromellose in the sustained release oral solid preparation.

Here, it is obvious that the sustained release excipient is incorporated in an amount of (100−x−y) wt % or less. The amount of the sustained release excipient is preferably 1 to 15 wt %, and more preferably 2 to 10 wt %, while satisfying the amount range of (100−x−y) wt % or less.

For example, when the sustained release oral solid preparation contains aripiprazole or a salt thereof in a proportion of 60 wt %, x of the above inequality is substituted with 60, and the following inequality is obtained:

27.02≦y≦33.02.

This indicates that the proportion of hypromellose is preferably 27.02 to 32.02 wt %.

When the sustained release oral solid preparation contains aripiprazole or a salt thereof in a proportion of about 45 to 85 wt % (preferably about 50 to 80 wt %), hypromellose is more preferably contained therein in a proportion satisfying the following inequality:

−0.733x+72≦y≦−0.733x+76,

wherein x and y are the same as above.

Examples of the method for producing the sustained release oral solid preparation of the present invention include a production method comprising mixing the above-mentioned sustained release excipient with aripiprazole or a salt thereof. Examples thereof also include a production method comprising preparing a sustained release excipient, and mixing the sustained release excipient with aripiprazole or a salt thereof to prepare a sustained release oral solid preparation.

A sustained release excipient and aripiprazole or a salt thereof can be mixed according to a known method. Specifically, a mixing method is exemplified in which a sustained release excipient and aripiprazole or a salt thereof (more preferably together with hypromellose) are placed in a mixer to be dry mixed, water is added thereto, and the mixture is mixed to obtain a granulation. Drying may further be performed after the granulation. The thus-obtained particles can be used as the sustained release oral solid preparation of the present invention.

The sustained release oral solid preparation obtained by the above-described production method has uniform packing characteristics over a wide range of various particle size distributions. Specifically, when the thus-obtained particles are filled within a certain volume, a high packing ratio can be achieved.

Further, the thus-obtained particles can be formulated into a preparation (e.g., tablets) after a lubricant is optionally added using, for example, direct compression or a conventional wet granulation. Such a preparation obtained in this manner can also be preferably used as the sustained release oral solid preparation of the present invention.

Specifically, in the step of “preparing a sustained release oral solid preparation” of the above-described production method, the form (dosage form) of the sustained release oral solid preparation is not particularly limited. For example, the step may only comprise mixing the sustained release excipient with aripiprazole or a salt thereof to obtain a mixed composition (e.g., granular material). Alternatively the step may comprise mixing the sustained release excipient with aripiprazole or a salt thereof to obtain a mixed composition, optionally adding a lubricant, etc., to the composition, and forming the resulting composition into a tablet or the like. It is also possible that the step further comprises, for example, forming a coating on the tablets.

As a more preferable embodiment of the production method of the present invention, a method comprising mixing a gelling agent, a cationic cross-linking agent, and an inert pharmaceutical diluent to obtain a sustained release excipient, adding aripiprazole or a salt thereof and hypromellose thereto, compressing the resulting mixture into tablets, and optionally enteric-coating the tablets can be exemplified.

The combination of the gelling agent (i.e., a mixture of xanthan gum and locust bean gum) with the inert diluent, with or without the cationic cross-linking agent and hypromellose, provides a ready-to-use product in which a formulator need only blend the desired active drug and an optional lubricant with the excipient and then compress the mixture to form slow-release tablets.

In particular, the combination of the cationic cross-linking agent, the gelling agent, and the inert diluent provides a ready-to-use excipient for a formulator. Such an excipient can be preferably used to release the drug in a sustained manner. For example, a formulator only need blend the desired active drug, hypromellose, if necessary, and an optional lubricant with the excipient, and then compress the mixture to form slow-release tablets. The slow-release tablets can provide sustained release, and can thus be used as a sustained release oral solid preparation.

The sustained release excipient may comprise a physical admix of the gum (xanthan gum and locust bean gum), an inert pharmaceutical diluent (e.g., saccharides usable as a soluble excipient, such as sucrose, lactose, dextrose, cellulose, mannitol, xylitol, and erythritol) and a cationic cross-linking agent. It is preferable to mix plain (i.e., crystalline) sucrose, lactose, dextrose, cellulose, mannitol, xylitol, erythritol, etc., as the inert pharmaceutical diluent, with the gums and the cationic cross-linking agent, and granulate or agglomerate the mixture to form the excipient. The granulate form has certain advantages including the fact that it can be optimized for flow and compressibility; it can be tableted, formulated in a capsule, extruded and spheronized with an active drug to form pellets, etc.

The sustained release excipients (in particular, pharmaceutically usable excipients) obtained, for example, in the above manner may be prepared according to any agglomeration technique to yield an acceptable excipient product. For example, in wet granulation techniques, the desired amounts of xanthan gum, locust bean gum, and the inert diluent are mixed together and thereafter a moistening agent such as water, propylene glycol, glycerol, alcohol or the like is added to prepare a moistened mass. Next, the moistened mass is dried. The dried mass is then milled with conventional equipment into granules to thereby obtain an excipient product. As such, the excipient product is ready to use.

The sustained release excipient is free-flowing and directly compressible. Accordingly, the excipient may be mixed in the desired proportion with aripiprazole or a salt thereof and optional lubricant (dry granulation). Alternatively, all or part of the excipient may be subjected to a wet granulation with aripiprazole or a salt thereof, and thereafter tableted. When the final product to be produced is tablets, the complete mixture, in an amount sufficient to make a uniform batch of tablets, is then subjected to tableting in a conventional production-scale tableting machine at normal compression pressure. However, the mixture should not be compressed to such a degree that there is subsequent difficulty in its hydration when exposed to gastric fluid.

One of the limitations of direct compression as a method of tablet production is the size of the tablet. If the amount of aripiprazole or a salt thereof is high, a formulator may choose to wet-granulate aripiprazole or a salt thereof with other excipients to attain a tablet of decent size with the right compact strength. Usually, the amount of filler/binder or excipients needed in wet granulation is less than that in direct compression since the process of wet granulation contributes to some extent toward the desired physical properties of a tablet.

The present invention is further related to a method for treating a central nervous system disease, comprising orally administering the above-described sustained release oral solid preparation to a patient.

The central nervous system disease treated by orally administering the above-described sustained release oral solid preparation to a patient include schizophrenia symptoms, manic symptoms in bipolar disorder, and disorders of the central nervous system associated with 5-HT_{1A} receptor subtype. Specific examples of disorders of the central nervous system associated with 5-HT_{1A} receptor subtype include the diseases disclosed in Japanese Patent No. 4178032. The disclosures of this patent document are incorporated herein by reference.

In particular, the treatment involving the oral administration of the sustained release oral solid preparation of the present invention is effective for Tourette's syndrome. It is preferable that the sustained release oral solid preparation of the present invention is administered, for example, as a once-weekly (QW) oral preparation, in order to improve medication compliance and QOL in pediatric patients.

Tourette's syndrome is a disease mainly developing in children (6 to 17 years old). The once-weekly (QW) oral preparation is possible by utilizing the long elimination half-life of the sustained release oral solid preparation of the present invention. In particular, with a high-dose administration, the effective blood level can be preferably maintained for about 1 week, up to preferably about 2 weeks.

The dosage of the above-described sustained release oral solid preparation is suitably selected depending on its usage, patient's age, sex, severity of the disease, and other conditions. In general, it is sufficient if the dose of the aripiprazole or a salt thereof, i.e., an active ingredient, is about 0.001 to 100 mg, preferably about 0.001 to 50 mg, per 1 kg of body weight per day. The frequency of the administration is suitably determined such that the effective blood level is maintained. For example, it is preferable that the administration be performed once per week.

The dosage of the above-described sustained release oral solid preparation in the treatment of Tourette's syndrome in pediatric patients is preferably administered once per week. The amount of aripiprazole or a salt thereof as the active ingredient is preferably about 10 to 200 mg/week, more preferably about 20 to 120 mg/week.

### EXAMPLES

The present invention is more specifically explained below with reference to Examples. The present invention is, however, not limited to these examples.

### Examples 1 to 3

Oral preparations (Tablets 1-3) were produced according to the following formulations and production processes.

### Example 1

### Tablet 1

| Aripiprazole | 48.5 wt % (52.5 mg) | |
|---|---|---|
| TIMERx ®-M50A | 9.7 | wt % |
| Hypromellose 2208 | 38.8 | wt % |
| Silicon dioxide | 0.5 | wt % |
| Sodium stearyl fumarate | 2.5 | wt % |

The tablets were formed using the above ingredients, and then coated with 6.5 mg of a methacrylic acid copolymer (Eudragit L30D-55).

### Example 2

### Tablet 2

| Aripiprazole | 58 wt % (77.5 mg) | |
|---|---|---|
| TIMERx ®-M50A | 8 | wt % |
| Hypromellose 2208 | 31 | wt % |
| Silicon dioxide | 0.5 | wt % |
| Sodium stearyl fumarate | 2.5 | wt % |

The tablets were formed using the above ingredients, and then coated with 8.0 mg of a methacrylic acid copolymer (Eudragit L30D-55).

### Example 3

### Tablet 3

| Aripiprazole | 66 wt % (110.0 mg) | |
|---|---|---|
| TIMERx ®-M50A | 6.0 | wt % |
| Hypromellose 2208 | 25 | wt % |
| Silicon dioxide | 0.5 | wt % |
| Sodium stearyl fumarate | 2.5 | wt % |

The tablets were formed using the above ingredients, and then coated with 10.0 mg of a methacrylic acid copolymer (Eudragit L30D-55).

Among the above ingredients, Hypromellose 2208 is produced by Dow Wolff, and TIMERx®-M50A (Penwest Pharmaceuticals) is a sustained release excipient produced according to the method disclosed in WO 2008/045060 (corresponding to Japanese PCT National-Phase Patent Publication No. 2010-505949). The disclosure of the above patent publication (in particular, Example 2) is hereby incorporated in the present specification by reference.

The respective proportions of the ingredients of TIMERx®-M50A are shown below.

| TIMERx ®-M50A | |
|---|---|
| Xanthan gum | 20 wt % |
| Locust bean gum | 30 wt % |
| Mannitol | 40 wt % |
| Calcium sulfate | 10 wt % |
| Water | 30-40* |

(*Water is removed during the process; i.e., water is removed during the manufacture of TIMERx®-M50A by mixing the above components. The above numerical range “30-40” indicates addition of 30-40 parts by weight of water relative to 100 parts by weight of the total of other components.)

The oral preparations (Tablets 1-3) of Examples 1 to 3 were produced according to the following process.

### Step 1: Dry Mixing

Tablets 1 to 3 are manufactured by first adding aripiprazole, TIMERx®-M50A and Hypromellose 2208 into a high shear mixer, and dry mixing the ingredients with the main impeller at low speed and the chopper turned off.

### Step 2: Granulation

Water is added at a constant flow rate to the mixer with the impeller and chopper at low speed. Following the water addition, the impeller and chopper are run at high speed until the desired granulation endpoint is reached.

### Step 3: Drying

The granulation is dried in a fluid bed dryer in two portions until the loss on drying (LOD) is less than 3%.

### Step 4: Size Reduction

The dried granulation is sized by passing it through a mill.

### Step 5: Blending

The granules are added to a V-blender along with the silicon dioxide (screened through a 30-mesh sieve), and blended for a set amount of time. The sodium stearyl fumarate (screened through a 30-mesh sieve) is added to the V-blender, and blended for a set amount of time.

### Step 6: Compression

The final blend is compressed on a tablet press to the desired weight, thickness, hardness, and friability specifications.

### Step 7: Film Coat

The enteric film coating is applied to the tablets by first preparing the coating dispersion. Talc and water are mixed until dispersed. Triethyl citrate is added to the talc dispersion, and mixed until dispersed. The talc dispersion is added to a Eudragit® L30D-55 dispersion (screened through 60-mesh sieve) while mixing until dispersed. The final dispersion is applied to the tablets in a coating pan until a weight gain of approximately 9.6% is reached. The coated tablets are cured in the coating pan at 35-45° C. for a minimum of 2 hours.

### Test Example 1

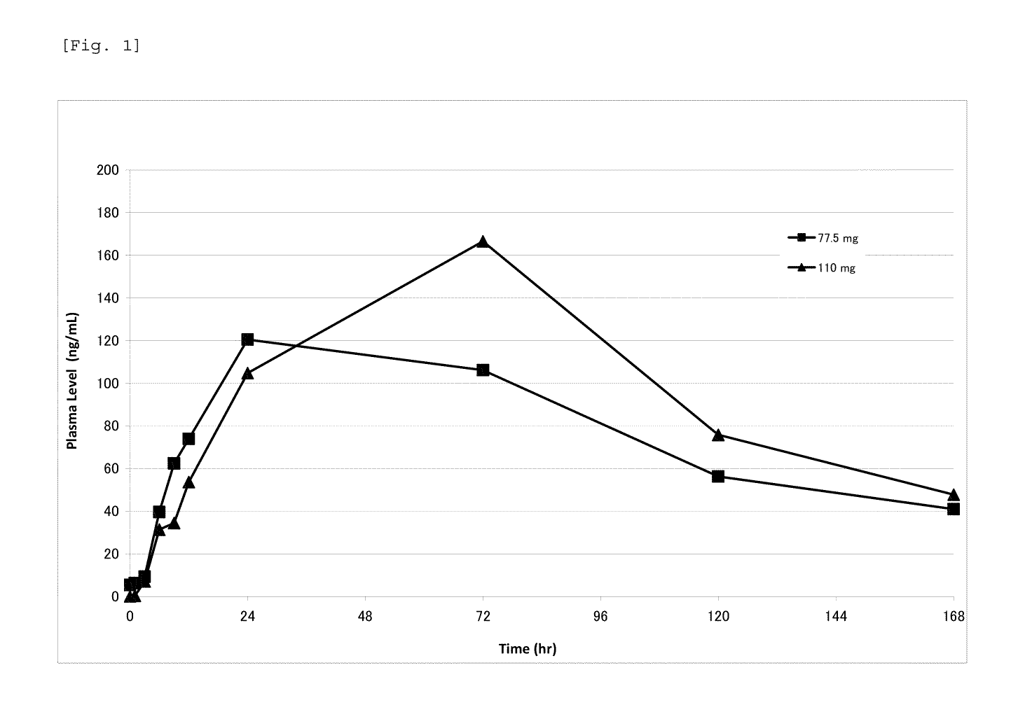

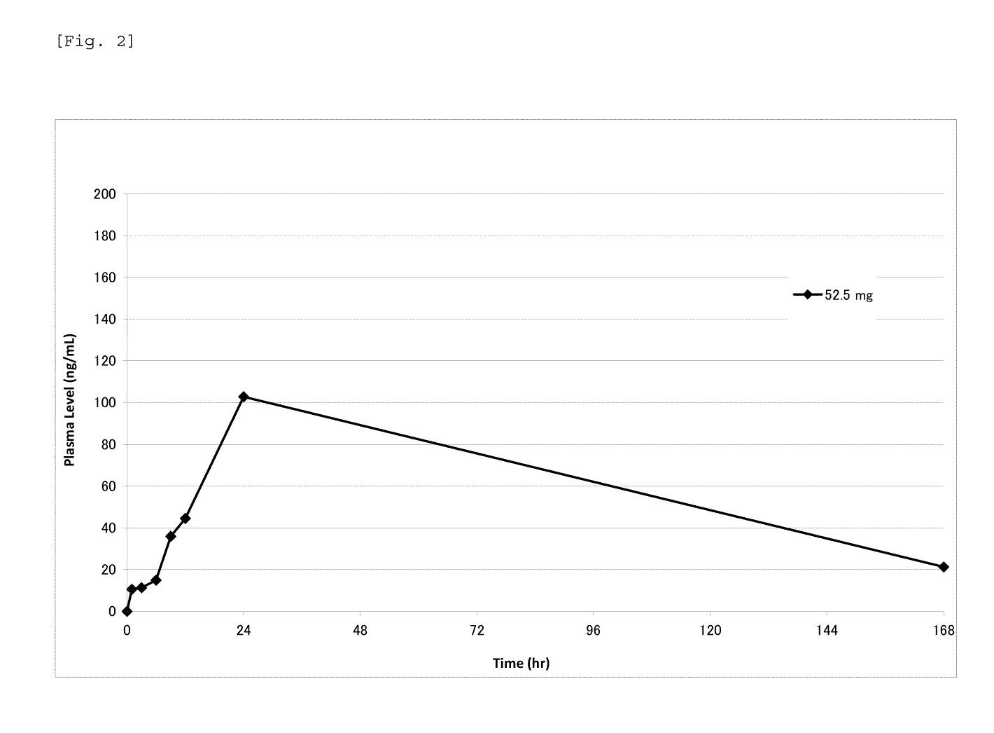

Oral Tablet 2 or 3 of Example 2 or 3 of the present invention was administered to fasted test subjects (pediatric Tourette's syndrome patients) (Tablet 2 (n=6), Tablet 3 (n=5)). FIG. 1 shows changes in blood level from the time of the administration to one week after the administration.

### Test Example 2

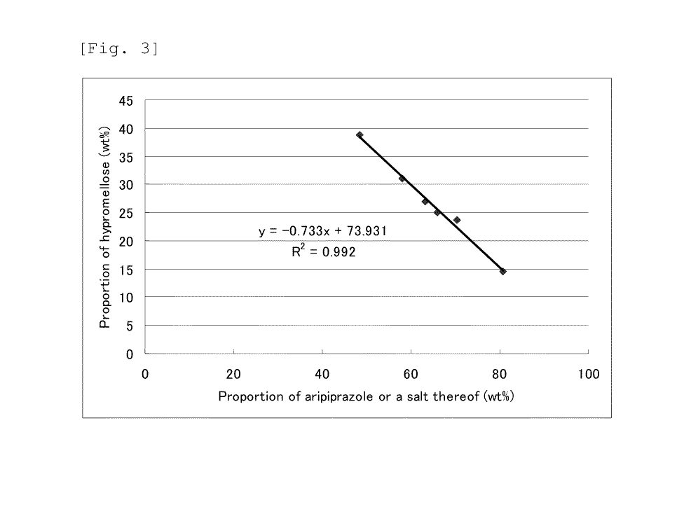

Oral Tablet 1 (n=6) of Example 1 was administered to test subjects (pediatric Tourette's syndrome patients) who were given a meal before the administration. FIG. 2 shows changes in blood level from the time of the administration to one week after the administration.

The results of Test Examples 1 and 2 showed that, even after a week, the sustained release oral solid preparation of the present invention satisfied the target PK profile of aripiprazole for Tourette's syndrome (in particular, pediatric Tourette's syndrome); that is, Cₘₐₓ≦150 ng/ml, C_{trough}≧20 ng/ml.

### Examples 4 to 6

The following oral preparations (Tablets 4 to 6) were produced according to the formulations below. As in the production methods of Tablets 1 to 3, Tablets 4 to 6 were produced by first producing core tablets through Steps 1 to 6, and then coating the core tablets (enteric coating and color film coating in this order). Tablets 4 to 6 were obtained as a result of analysis of effective formulations of the components to produce preparations exhibiting an excellent sustained-release characteristic of aripiprazole. In particular, the preparations are considered to be effective for schizophrenia.

### Example 4

### Tablet 4

TABLE 1
Per Tablet (266.0 mg)

| | | | | | Amount | |
|---|---|---|---|---|---|---|
| | | Material Name | Product Name/Grade | Manufacturer (Retailer) | (mg) | Purpose of Incorporation |
| Components of | 1 | Aripiprazole | Anhydride Pulverized by | Otsuka Pharmaceutical Co., Ltd. | 150.0 | Main Component |
| Core Tablet | | | Hammer Mill | | | |
| (Uncoated | 2 | TIMERx ®-M50A*¹ | TIMERx ®-M50A*¹ | Penwest*² | 16.0 | Sustained Release Base |
| Tablet) | 3 | Hypromellose2208 | Methocel K100 Premium LV | Dow Wolff | 64.0 | Sustained Release Base |
| | 4 | Purified Water*³ | Purified Water | Otsuka Pharmaceutical Co., Ltd. | (103.5) | Binder Liquid |
| | 5 | Silicon Dioxide*⁴ | Syroid 244FP | Grace | 1.2 | Superplasticizer |
| | 6 | Sodium Stearyl | PRUV | JRS Pharma | 5.9 | Lubricant |
| | | Fumarate*⁴ | | | | |
| | Total Solids Content of Core Tablet | | | | 237.1 | |
| Components of | 7 | Methacrylic Acid | Eudragit L30D-55 | Evonik Degussa | 12.3*⁵ | Coating Agent |
| Enteric Film | | Copolymer Dispersion | | | | |
| Coating | 8 | Talc | Talc PKP-81 | Fuji Talc Industrial Co., Ltd. | 6.2 | Lubricant |
| | 9 | Triethyl Citrate | Citroflex No. 2 (SC-60) | Morimura Bros., Inc. | 1.2 | Plasticizer |
| | 10 | Purified Water*³ | Purified Water | Otsuka Pharmaceutical Co., Ltd. | (50.1) | Solvent of Coating Liquid |
| | Total Solids Content of Enteric Film | | | | 19.7 | |
| | Total Solids Content of Enteric Film Coated Tablet | | | | 256.8 | |
| Components of | 11 | OPADRY 03A420003 | OPADRY 03A420003 Yellow | Colorcon | 9.2 | Coating Agent |
| Color Film | | Yellow*⁶ | | | | |
| Coating | 12 | Purified Water*³ | Purified Water | Otsuka Pharmaceutical Co., Ltd. | (82.8) | Solvent of Coating Liquid |

| | |
|---|---|
| Total Solids Content of Color Film | 9.2 |
| Total Solids Content of Color Film Coated Tablet | 266.0 |

*¹Same as the one used in Examples 1 to 3.
*²Currently known as Endo Pharmaceuticals.
*³The amount can be changed within an appropriate range; water is completely removed during the core tablet production step or the coating step.
*⁴Silicon dioxide is added after the granulation step as in the manufacture of Tablets 1 to 3 (corresponding to Step 5 in the methods of Examples 1 to 3).
*⁵Solids content weight in Eudragit L30D-55.
*⁶Hypromellose 2910 (6 cp): 67.00%, Titanium dioxide: 31.09%, Iron oxide yellow: 1.91%

### Example 5

### Tablet 5

TABLE 2
Per Tablet (386.6 mg)

| | | | | | Amount | |
|---|---|---|---|---|---|---|
| | | Material Name | Product Name/Grade | Manufacturer (Retailer) | (mg) | Purpose of Incorporation |
| Components of | 1 | Aripiprazole | Anhydride Pulverized by | Otsuka Pharmaceutical Co., Ltd. | 250.0 | Main Component |
| Core Tablet | | | Hammer Mill | | | |
| (Uncoated | 2 | TIMERx ®-M50A*¹ | TIMERx ®-M50A*¹ | Penwest*² | 10.5 | Sustained Release Base |
| Tablet) | 3 | Hypromellose2208 | Methocel K100 Premium LV | Dow Wolff | 84.0 | Sustained Release Base |
| | 4 | Purified Water*³ | Purified Water | Otsuka Pharmaceutical Co., Ltd. | (158.47) | Binder Liquid |
| | 5 | Silicon Dioxide*⁴ | Syroid 244FP | Grace | 1.8 | Superplasticizer |
| | 6 | Sodium Stearyl | PRUV | JRS Pharma | 8.9 | Lubricant |
| | | Fumarate*⁴ | | | | |
| | Total Solids Content of Core Tablet | | | | 355.2 | |
| Components of | 7 | Methacrylic Acid | Eudragit L30D-55 | Evonik Degussa | 13.4*⁵ | Coating Agent |
| Enteric Film | | Copolymer Dispersion | | | | |
| Coating | 8 | Talc | Talc PKP-81 | Fuji Talc Industrial Co., Ltd | 6.7 | Lubricant |
| | 9 | Triethyl Citrate | Citroflex No. 2 (SC-60) | Morimura Bros., Inc. | 1.3 | Plasticizer |
| | 10 | Purified Water*³ | Purified Water | Otsuka Pharmaceutical Co., Ltd. | (54.33) | Solvent of Coating Liquid |
| | Total Solids Content of Enteric Film | | | | 21.4 | |
| | Total Solids Content of Enteric Film Coated Tablet | | | | 376.6 | |
| Components of | 11 | OPADRY 03A420003 | OPADRY 03A420003 Yellow | Colorcon | 10.0 | Coating Agent |
| Color Film | | Yellow*⁶ | | | | |
| Coating | 12 | Purified Water*³ | Purified Water | Otsuka Pharmaceutical Co., Ltd. | (90.0) | Solvent of Coating Liquid |

| | |
|---|---|
| Total Solids Content of Color Film | 10.0 |
| Total Solids Content of Color Film Coated Tablet | 386.6 |

*¹Same as the one used in Examples 1 to 3.
*²Currently known as Endo Pharmaceuticals.
*³The amount can be changed within an appropriate range; water is completely removed during the core tablet production step or the coating step.
*⁴Silicon dioxideis added after the granulation step as in the manufacture of Tablets 1 to 3. (corresponding to Step 5 in the methods of Examples 1 to 3).
*⁵solids content weight in Eudragit L30D-55.
*⁶Hypromellose 2910 (6 cp): 67.00%, Titanium dioxide: 31.09%, Iron oxide yellow: 1.91%

### Example 6

### Tablet 6

TABLE 3
Per Tablet (476.3 mg)

| | | | | | Amount | |
|---|---|---|---|---|---|---|
| | | Material Name | Product Name/Grade | Manufacturer (Retailer) | (mg) | Purpose of Incorporation |
| Components of | 1 | OPC-14597 | Anhydride Pulverized by | Otsuka Pharmaceutical Co., Ltd. | 350.0 | Main Component |
| Core Tablet | | | Hammer Mill | | | |
| (Uncoated | 2 | TIMERx ®-M50A*¹ | TIMERx ®-M50A*¹ | Penwest*² | 7.0 | Sustained Release Base |
| Tablet) | 3 | Hypromellose2208 | Methocel K100 Premium LV | Dow Wolff | 63.0 | Sustained Release Base |
| | 4 | Purified Water*³ | Purified Water | Otsuka Pharmaceutical Co., Ltd. | (189.0) | Binder Liquid |
| | 5 | Silicon Dioxide*⁴ | Syroid 244FP | Grace | 2.2 | Superplasticizer |
| | 6 | Sodium Stearvl | PRUV | JRS Pharma | 10.8 | Lubricant |
| | | Fumarate*⁴ | | | | |
| | Total Solids Content of Core Tablet | | | | 433.0 | |
| Components of | 7 | Methacrylic Acid | Eudragit L30D-55 | Evonik Degussa | 18.4*⁵ | Coating Agent |
| Enteric Film | | Copolymer Dispersion | | | | |
| Coating | 8 | Talc | Talc PKP-81 | Fuji Talc Industrial Co., Ltd | 9.2 | Lubricant |
| | 9 | Triethyl Citrate | Citroflex No. 2 (SC-60) | Morimura Bros., Inc. | 1.9 | Plasticizer |
| | 10 | Purified Water*³ | Purified Water | Otsuka Pharmaceutical Co., Ltd. | (75.07) | Solvent of Coating Liquid |
| | Total Solids Content of Enteric Film | | | | 29.5 | |
| | Total Solids Content of Enteric Film Coated Tablet | | | | 462.5 | |
| Components of | 11 | OPADRY 03A420003 | OPADRY 03A420003 Yellow | Colorcon | 13.8 | Coating Agent |
| Color Film | | Yellow*⁶ | | | | |
| Coating | 12 | Purified Water*³ | Purified Water | Otsuka Pharmaceutical Co., Ltd. | (124.2) | Solvent of Coating Liquid |

| | |
|---|---|
| Total Solids Content of Color Film | 13.8 |
| Total Solids Content of Color Film Coated Tablet | 476.3 |

*¹Same as the one used in Examples 1 to 3.
*²Currently known as Endo Pharmaceuticals.
*³The amount can be changed within an appropriate range; water is completely removed during the core tablet production step or the coating step.
*⁴Silicon dioxide is added after the granulation step as in the manufacture of Tablets 1 to 3. (corresponding to Step 5 in the methods of Examples 1 to 3).
*⁵Solids content weight in Eudragit L30D-55.
*⁶Hypromellose 2910 (6 cp): 67.00%, Titanium dioxide: 31.09%, Iron oxide yellow: 1.91%

Table 4 summarizes proportions (wt %) of aripiprazole or salts thereof, and hypromellose in the sustained release oral solid preparations (uncoated tablets) in Examples 1 to 6 (Tablets 1 to 6). FIG. 3 shows a linear approximation graph based on Table 4.

TABLE 4

| | Example 1 | Example 2 | Example 3 | Example 4 | Example 5 | Example 6 |
|---|---|---|---|---|---|---|
| x (proportion | 48.5 | 58 | 66 | 63 | 70.4 | 80.8 |
| (wt %) of | | | | | | |
| aripiprazole or a | | | | | | |
| salt thereof) | | | | | | |
| y (proportion | 38.8 | 31 | 25 | 27 | 23.6 | 14.5 |
| (wt %) of | | | | | | |
| hypromellose) | | | | | | |

According to FIG. 3, found was an approximate equation: y=−0.733x+73.931, wherein proportion (wt %) of aripiprazole or a salt thereof in the sustained release oral solid preparation (uncoated tablet) is x, and proportion (wt %) of hypromellose in the sustained release oral solid preparation (uncoated tablet) is y. It was also confirmed that the accuracy was very high (R²=0.992).

Further, this also revealed that, in order to obtain a preparation exhibiting an excellent sustained-release characteristic of aripiprazole, there is the above regularity between proportion (wt %) of aripiprazole or a salt thereof and proportion (wt %) of hypromellose in the preparation of the present invention. Therefore, it was concluded that the preparation is preferably produced by ensuring the regularity.

More specifically, it was concluded that it is particularly preferable to incorporate hypromellose in an amount satisfying the following inequality when the sustained release oral solid preparation contains about 45 to 85 wt % of aripiprazole or a salt thereof.

−0.733x+71≦y≦−0.733x+77

wherein x represents a proportion of aripiprazole or a salt thereof (wt %) in the sustained release oral solid preparation, and y represents a proportion of hypromellose (wt %) in the sustained release oral solid preparation.

The above inequality defines a range about ±3 of the value y in the above approximate equation (y=−0.733x+73.931). In all of Examples 1 to 6, the proportions (wt %) of aripiprazole or salts thereof and the proportions (wt %) of hypromellose satisfy this range.

1. A sustained release oral solid preparation comprising aripiprazole or a salt thereof, and a sustained release excipient,
the sustained release excipient comprising
a gelling agent comprising xanthan gum and locust bean gum;
at least one inert pharmaceutical diluent selected from the group consisting of monosaccharides, disaccharides, polyhydric alcohols, and mixtures thereof; and
a pharmaceutically acceptable cationic cross-linking agent capable of crosslinking with the gelling agent and increasing the gel strength when the sustained release oral solid preparation is exposed to an environmental fluid,
the ratio of the xanthan gum to the locust bean gum in the gelling agent being about 1:1 to 1:3 by weight,
the ratio of the inert pharmaceutical diluent to the gelling agent being about 1:1 to 1:2 by weight.

2. The sustained release oral solid preparation according to claim 1, further comprising hypromellose, wherein the ratio of the hypromellose to the aripiprazole or a salt thereof is about 1:0.1 to 1:5.

3. The sustained release oral solid preparation according to claim 1, wherein the cationic cross-linking agent is at least one salt selected from the group consisting of sulfate, chloride, borate, carbonate, phosphate, bromide, citrate, acetate, and lactate, and wherein the salt is an alkali metal salt or an alkaline earth metal salt.

4. The sustained release oral solid preparation according to claim 1, wherein the cationic cross-linking agent comprises calcium sulfate.

5. A sustained release oral solid preparation comprising aripiprazole or a salt thereof, and a sustained release excipient,
the sustained release excipient comprising
a gum comprising a combination of xanthan gum with locust bean gum;
a sugar alcohol; and
an inorganic or organic salt of an alkali metal and/or an alkaline earth metal,
the ratio of the xanthan gum to the locust bean gum being about 1:1 to 1:3 by weight, and
the ratio of the sugar alcohol to the gum being within the range of about 1:1 to 1:2 by weight.

6. The sustained release oral solid preparation according to claim 5, further comprising hypromellose.

7. The sustained release oral solid preparation according to claim 6, wherein the sustained release oral solid preparation comprises about 45 to 85 weight % of aripiprazole or a salt thereof, and hypromellose in an amount satisfying the following inequality:
−0.733x+71≦y≦−0.733x+77,
wherein x represents a proportion (wt %) of the aripiprazole or a salt thereof, and y represents a proportion (wt %) of the hypromellose.

8. The sustained release oral solid preparation according to claim 1, which is a tablet.

9. The sustained release oral solid preparation according to claim 1, wherein at least part of a surface of the sustained release oral solid preparation is coated with an enteric material, and wherein the sustained release oral solid preparation coated with the enteric material has a weight increased by about 1 to 20 wt %, relative to the sustained release oral solid preparation before coating.

10. The sustained release oral solid preparation according to claim 9, wherein the enteric material comprises a methacrylic acid copolymer.

11. The sustained release oral solid preparation according to claim 1, wherein the content of the cationic cross-linking agent is about 0.5 to 20 wt % of the sustained release excipient.

12. A method for producing a sustained release oral solid preparation, comprising
mixing aripiprazole or a salt thereof with a sustained release excipient,
the sustained release excipient comprising
about 3.0 to 98.5 wt % of a gum comprising xanthan gum and locust bean gum;
about 1.0 to 89 wt % of a sugar alcohol; and
about 0.5 to 20 wt % of an inorganic or organic salt of an alkali metal and/or an alkaline earth metal,
the ratio of the xanthan gum to the locust bean gum being about 1:1 to 1:3 by weight, and
the ratio of the sugar alcohol to the gum being within the range of about 1:1 to 1:2 by weight.

13. A method for producing a sustained release oral solid preparation, comprising:
preparing a sustained release excipient comprising
about 3.0 to 98.5 wt % of a gelling agent comprising xanthan gum and locust bean gum;
about 1.0 to 89 wt % of an inert pharmaceutical diluent; and
about 0.5 to 20 wt % of a cationic cross-linking agent capable of crosslinking with the gelling agent and increasing the gel strength when the sustained release oral solid preparation is exposed to an environmental fluid,
the ratio of the xanthan gum to the locust bean gum being about 1:1 to 1:3 by weight; and
mixing the sustained release excipient with aripiprazole or a salt thereof to prepare a sustained release oral solid preparation,
wherein when the sustained release oral solid preparation is exposed to an environmental fluid, the preparation maintains a therapeutically effective blood level of the aripiprazole or a salt thereof for at least 12 hours.

14. The method for producing a sustained release oral solid preparation according to claim 12, wherein the sustained release excipient is mixed with aripiprazole or a salt thereof so that the produced sustained release oral solid preparation comprises about 45 to 85 wt % of aripiprazole or a salt thereof, and hypromellose in an amount satisfying the following inequality:
−0.733x+71≦y≦−0.733x+77,
wherein x represents the proportion (wt %) of the aripiprazole or a salt thereof, and y represents the proportion (wt %) of the hypromellose.

15. A method for treating a central nervous system disease, comprising orally administering the sustained release oral solid preparation of claim 1 to a patient suffering from a central nervous system disease.

16. The sustained release oral solid preparation according to claim 5, which is a tablet.

17. The sustained release oral solid preparation according to claim 5, wherein at least part of a surface of the sustained release oral solid preparation is coated with an enteric material, and wherein the sustained release oral solid preparation coated with the enteric material has a weight increased by about 1 to 20 wt %, relative to the sustained release oral solid preparation before coating.

18. The sustained release oral solid preparation according to claim 17, wherein the enteric material comprises a methacrylic acid copolymer.

19. The method for producing a sustained release oral solid preparation according to claim 13, wherein the sustained release excipient is mixed with aripiprazole or a salt thereof so that the produced sustained release oral solid preparation comprises about 45 to 85 wt % of aripiprazole or a salt thereof, and hypromellose in an amount satisfying the following inequality:
−0.733x+71≦y≦−0.733x+77,
wherein x represents the proportion (wt %) of the aripiprazole or a salt thereof, and y represents the proportion (wt %) of the hypromellose.

20. A method for treating a central nervous system disease, comprising orally administering the sustained release oral solid preparation of claim 5 to a patient suffering from a central nervous system disease.
